(19) United States
(12) Patent Application Publication
(10) Pub. No.: US 2011/0229545 A1
(43) Pub. Date: Sep. 22, 2011

(54) MELT EMULSIFICATION

(75) Inventors: Ho Cheung Shum, Hong Kong (CN); Bingjie Sun, Shanghai (CN); David A. Weitz, Bolton, MA (US); Christian Holtze, Frankfurt (DE)

(73) Assignees: President and Fellows of Harvard College, Cambridge, MA (US); BASF SE, Ludwigshafen (DE)

(21) Appl. No.: 13/049,957

(22) Filed: Mar. 17, 2011

Related U.S. Application Data
(60) Provisional application No. 61/314,841, filed on Mar. 17, 2010.

(51) Int. Cl. A01N 25/08; A01P 15/00; A61K 9/54; B82Y 5/00

(52) U.S. Cl. 424/409; 264/4.32; 977/773

(57) ABSTRACT

The present invention generally relates to colloidal systems, which may include colloidal particles and/or other types of particles. One aspect of the invention is generally directed to a system comprising fluidic droplets that can be at least partially solidified, e.g., to form colloidal particles. In some embodiments, particles comprising an at least partially solid outer phase encapsulating an inner phase are formed. The inner phase may be any phase, e.g., a solid, a liquid, or a gas. In some cases, solidifying at least a portion of the outer phase of the droplets to form particles may increase the stability of the particles and/or the colloidal system containing the particles. In one set of embodiments, melting or liquefying the outer phase of the particles (for example, by heating the particle to a temperature above a threshold temperature) can allow release of an agent contained within the inner phase, and/or allow the inner phase to coalesce with a phase external to the particles. The melting temperature of the outer phase can be controlled in some embodiments such that the outer phase will melt above a predetermined temperature. In some embodiments, the particles may be formed to be essentially free of an auxiliary stabilizing agent. In some embodiments, an agent may be encapsulated within a particle with relatively high efficiency. Other aspects of the invention are generally directed to methods of making and using such colloidal systems, e.g., containing such particles, kits involving such colloidal systems, or the like.

### RELATED APPLICATIONS

This application claims the benefit of U.S. Provisional Patent Application Ser. No. 61/314,841, filed Mar. 17, 2010, entitled “Melt Emulsification,” by Shum, et al., incorporated herein by reference.

### GOVERNMENT FUNDING

Research leading to various aspects of the present invention were sponsored, at least in part, by the National Science Foundation, Grant Nos. DMR-0820484 and DMR-0602684. The U.S. Government has certain rights in the invention.

### FIELD OF INVENTION

The present invention generally relates to colloidal and other systems, which may include colloidal particles and/or other types of particles.

### BACKGROUND

Colloidal systems generally involve a first phase dispersed within a second phase. For example, one type of colloidal system is a fluidic state which exists when a first fluid is dispersed in a second fluid that is typically immiscible with the first fluid (e.g., an emulsion). Examples of common emulsions are oil in water and water in oil emulsions. Multiple emulsions are emulsions that are formed with more than two fluids, and/or with two or more fluids arranged in a more complex manner than a typical two-fluid emulsion. For example, a multiple emulsion may be an oil-in-water-in-oil emulsion (“o/w/o”), or a water-in-oil-in-water emulsion (“w/o/w”). Multiple emulsions are of particular interest because of current and potential applications in fields such as pharmaceutical delivery, paints, inks and coatings, food and beverage, chemical separations, and health and beauty aids.

Typically, multiple emulsions of a droplet inside another droplet are made using a two-stage emulsification technique, which may include applying shear forces through mixing to reduce the size of droplets formed during the emulsification process. Other methods, such as membrane emulsification techniques using, for example, a porous glass membrane, have also been used to produce water-in-oil-in-water emulsions. Microfluidic techniques have also been used to produce droplets inside of droplets using a procedure including two or more steps. For example, see International Patent Application No. PCT/US2004/010903, filed Apr. 9, 2004, entitled “Formation and Control of Fluidic Species,” by Link, et al., published as WO 2004/091763 on Oct. 28, 2004; or International Patent Application No. PCT/US03/20542, filed Jun. 30, 2003, entitled “Method and Apparatus for Fluid Dispersion,” by Stone, et al., published as WO 2004/002627 on Jan. 8, 2004, each of which is incorporated herein by reference.

### SUMMARY OF THE INVENTION

The present invention generally relates to colloidal systems, which may include colloidal particles and/or other types of particles. The subject matter of the present invention involves, in some cases, interrelated products, alternative solutions to a particular problem, and/or a plurality of different uses of one or more systems and/or articles.

One aspect of the invention is generally directed to a colloidal particle comprising an at least partially solid outer phase and an inner phase, the at least partially solid outer phase encapsulating the inner phase, wherein the at least partially solid outer phase has a melting temperature greater than 0° C.

In another aspect, the present invention is generally directed to a particle having an average diameter of less than about 1 mm. In some embodiments, the particle comprises an at least partially solid outer phase and an inner phase. In certain cases, the at least partially solid outer phase partially or completely encapsulates the inner phase. In some instances, the at least partially solid outer phase has a melting temperature greater than about 0° C.

Still another aspect of the invention is generally directed to a method comprising combining an outer fluid with an inner phase, the inner phase comprising an agent; and forming a multiple emulsion, wherein at least 90% of the agent is encapsulated in a droplet of the multiple emulsion.

In another aspect of the invention, the method is generally directed to acts of providing a first fluid and a second fluid, surrounding at least portion of the second fluid with the first fluid to form a multiple emulsion, and solidifying at least a portion of the first fluid to form a capsule. In certain embodiments, the second fluid comprises a species, and in some cases, at least 90% of the species is partially or completely encapsulated within the capsule. In one set of embodiments, the first fluid and the second fluid may be at least partially immiscible.

Yet another aspect of the invention is generally directed to a method comprising providing a droplet comprising an outer phase and an inner phase, the outer phase encapsulating the inner phase, wherein the outer phase has a melting temperature greater than 0° C.; and solidifying at least a portion of the outer phase by altering temperature.

The method, in accordance with another aspect, includes acts of providing a droplet having an average diameter of less than about 1 mm, and solidifying at least a portion of the outer phase by altering the temperature of the droplet to produce a capsule. In some embodiments, the droplet comprises an outer phase and an inner phase, and in some cases, the outer phase partially or completely encapsulates the inner phase. In certain instances, the outer phase has a melting temperature greater than 0° C.

Still another aspect of the invention is generally directed to a method comprising providing a colloidal particle comprising an at least partially solid outer phase and an inner phase, the at least partially solid outer phase encapsulating the inner phase, wherein the at least partially solid outer phase has a melting temperature greater than 0° C.; and releasing an agent from the colloidal particle, wherein releasing the agent from the droplet comprises melting the at least partially solid outer phase.

The method, in yet another aspect of the invention, includes acts of providing a particle having an average diameter of less than about 1 mm, and releasing a species from the particle by melting the at least partially solid outer phase. According to some embodiments, the particle comprises an at least partially solid outer phase and an inner phase, and in some cases, the at least partially solid outer phase partially or completely encapsulates the inner phase. In one set of embodiments, the at least partially solid outer phase has a melting temperature greater than 0° C.

Another aspect of the invention is directed to a particle having a shell surrounding a liquid core, the shell having a melting temperature greater than about 0° C. In another aspect, the invention is generally directed to a particle having a shell surrounding at least one liquid core. In some embodiments, the particle has an average diameter of less than about 1 mm. In certain cases, the shell has a melting temperature greater than about 0° C.

Yet another aspect of the invention is directed to a method comprising exposing a multiple emulsion comprising an inner fluid and an outer fluid to an external temperature and pressure such that at least a portion of the outer fluid reversibly solidifies. The invention, in accordance with another aspect, includes an act of exposing a multiple emulsion droplet comprising an inner fluidic droplet and an outer fluidic droplet to external temperature and/or pressure such that at least a portion of the outer fluidic droplet solidifies.

Still another aspect of the invention is directed to a method comprising providing a multiple emulsion comprising an inner fluid and an outer fluid at a first temperature and a first pressure; and exposing the multiple emulsion to a second temperature and a second pressure sufficient to at least partially solidify one of the inner fluid and the outer fluid, wherein at least one of (1) the first temperature and the second temperature are different, or (2) the first pressure and the second pressure are different, wherein, after exposing the multiple emulsion to a second temperature and a second pressure, exposure of the multiple emulsion to the first temperature and a first pressure causes the solidified portion of the multiple emulsion to melt.

The method, in accordance with yet another aspect, includes acts of providing a multiple emulsion droplet comprising an inner fluidic droplet and an outer fluidic droplet at a first temperature and a first pressure, and exposing the multiple emulsion droplet to a second temperature and/or a second pressure sufficient to at least partially solidify one of the inner fluidic droplet and the outer fluidic droplet. In some cases, the first temperature and the second temperature are different, and/or the first pressure and the second pressure are different. In certain embodiments, after exposing the multiple emulsion droplet to a second temperature and/or a second pressure, exposure of the multiple emulsion droplet to the first temperature and the first pressure is able to cause the solidified portion of the multiple emulsion droplet to melt.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A shows the formation of double emulsion droplets in a microfluidic device using a flow focusing geometry, according to certain embodiments of the invention;

FIG. 1B shows a schematic of a microfluidic device, according to another embodiment of the invention;

FIG. 1C shows a schematic illustrating the encapsulation and release of one or more species from particles formed from double emulsion droplets, according to an embodiment of the invention;

FIG. 2A shows a bright field microscope image of particles having solid shells comprising fatty acid glycerides, according to one embodiment of the invention;

FIG. 2B shows a fluorescence microscope image of the same area as in FIG. 2A;

FIG. 2C shows a bright-field microscope image of particles, formed from double emulsion droplets, having solid shells of fatty acid glycerides, after the particles were stored at room temperature for 6 months, according to another embodiment of the invention;

FIG. 2D shows a fluorescence microscope image of the same area as in FIG. 2C;

FIG. 3A illustrates a sequence of fluorescence microscope images showing the release of fluorescent beads from particles of fatty acid glycerides, according to one embodiment of the invention;

FIG. 3B illustrates a sequence of bright field microscope images showing the release of toluidine blue from particles of paraffin, according to another embodiment of the invention;

FIGS. 3C-3D illustrates a sequence of images showing the release of laundry detergent from particles of fatty acid glycerides mixed with hexadecane, according to an embodiment of the invention;

FIGS. 4A-4E show certain particles having various shells encapsulating various species, according to certain embodiments of the invention;

FIG. 5A shows a schematic of a micro fluidic device for generating two-bore double emulsion droplets, according to certain embodiments of the invention;

FIG. 5B shows a bright field microscope image of particles having two inner compartments respectively containing aqueous solutions of Wright stain (light) and rhodamine B (dark), according to some embodiments of the invention;

FIG. 5C illustrates an SEM (scanning electron microscopy) image of a dried particle from FIG. 5B, showing the surface of the particle; and

FIG. 5D illustrates an SEM image of a particle from FIG. 5B, showing a cross-section of the particle.

### DETAILED DESCRIPTION

The present invention generally relates to colloidal systems, which may include colloidal particles and/or other types of particles. One aspect of the invention is generally directed to a system comprising fluidic droplets that can be at least partially solidified, e.g., to form particles. In some embodiments, particles comprising an at least partially solid outer phase encapsulating an inner phase are formed. The inner phase may be any phase, e.g., a solid, a liquid, or a gas. In some cases, solidifying at least a portion of the outer phase of the droplets to form particles may increase the stability of the particles and/or the colloidal system containing the particles. In one set of embodiments, melting or liquefying the outer phase of the particles can allow release of a species contained within the inner phase, and/or allow the inner phase to coalesce with a phase external to the particles. The melting temperature of the outer phase can be controlled in some embodiments such that the outer phase will melt above a predetermined temperature. In some embodiments, the particles may be formed to be essentially free of an auxiliary stabilizing agent. In some embodiments, a species may be encapsulated within a particle with relatively high efficiency. Other aspects of the invention are generally directed to methods of making and using such colloidal systems, e.g., containing such particles, kits involving such colloidal systems, or the like.

A colloidal system generally includes at least two separate phases, a dispersed, first phase and a continuous, second phase. One example of a colloidal system is an emulsion, in which the dispersed phase (e.g., a first fluid) and the continuous phase (e.g., a second fluid) of the colloidal system are both liquids. Another example of a colloidal system is a particle suspension, in which the dispersed phase is solid and the continuous phase is liquid. In some embodiments, the particles in the particle suspension may include a liquid phase or other fluid phase inside the solid particle, as discussed in more detail below. In one embodiment, a dispersed, first fluid contained within a continuous, second fluid of an emulsion (i.e., dispersed as droplets therein) may be at least partially solidified, using techniques such as those discussed herein, to form a particle suspension.

In certain embodiments, the present invention generally relates to emulsions, including multiple emulsions, and to methods and apparatuses for making or using such emulsions. A “multiple emulsion,” as used herein, describes a system of larger fluidic droplets that contain one or more smaller fluidic droplets therein, which in turn may contain even smaller fluidic droplets within those droplets, and this may be repeated any number of times. In a double emulsion, for example, a continuous fluid may contain one or more droplets therein, which may, in turn, contain one or more smaller droplets therein. The smaller droplets may contain the same or different fluids than the continuous fluid containing the droplets. In certain embodiments, larger degrees of nesting of droplets-within-droplets within the multiple emulsion are possible. Multiple emulsions can be useful for encapsulating species such as pharmaceutical agents, cells, chemicals, or the like. As described below, multiple emulsions can be formed in certain embodiments with generally precise repeatability. In some cases, the encapsulation of a species may be performed relatively quantitatively, as discussed herein.

A multiple emulsion may be thermodynamically unstable, at least in some instances, and the droplets contained within a multiple emulsion can disintegrate under certain circumstances by contact or coalescence with other, miscible droplets. However, the stability of droplets contained within a multiple emulsion can be improved, for example, by forming the multiple emulsion in the presence of an auxiliary stabilizing agent (e.g., a surfactant). Examples of auxiliary stabilizing agents are discussed below. In some embodiments, the droplets within a multiple emulsion can be stabilized by at least partially solidifying the droplets, or portions of the droplets, to form a particle suspension where coalescence of the droplets is at least partially inhibited due to the formation of a solid phase. Furthermore, according to some embodiments, returning the solidified portions of the particles to a liquid phase (e.g., by melting the solidified portions) can reintroduce the instability. Accordingly, in some embodiments of the invention as disclosed herein, a phase change in a particle can accordingly be used to trigger release of one or more species from the particle. As discussed in more detail below, a multiple emulsion can be formed using a continuous phase fluid by combining an outer phase fluid and an inner phase fluid within the continuous phase fluid, where the outer phase fluid encapsulates the inner phase fluid, i.e., the outer phase fluid partially or completely surrounds the inner phase fluid. The encapsulation may be complete or partial.

In some embodiments, a colloidal system such as a multiple emulsion may be one that is essentially free of an auxiliary stabilizing agent. An auxiliary stabilizing agent is an agent, such as a surfactant, that when added to a multiple emulsion, increases the stability of the multiple emulsion relative to when no such auxiliary stabilizing agent is added. For example, the stability of the multiple emulsion may increase such that a relatively longer amount of time is needed for the multiple emulsion to disintegrate by droplet coalescence such that the resultant system can no longer be considered to be a multiple emulsion. For instance, the amount of time may be at least 2-fold, at least 3-fold, at least 5-fold, at least 10-fold, at least 30-fold, at least 50-fold, or at least 100-fold. In some cases, the auxiliary stabilizing agent may stabilize the multiple emulsion droplet such that no droplet instability or coalescence can be detected, e.g., for at least a period of at least about a day or a week. Non-limiting examples of auxiliary stabilizing agents include sorbiton-monooleate or sodium dodecyl sulfate.

As used herein, “essentially free of auxiliary stabilizing agent,” in reference to a particle containing a solid outer phase and a non-solid inner phase containing a species, means that the particles does not contain an auxiliary stabilizing agent, or contains less auxiliary stabilizing agent than is needed to prevent release of the species from the particle when the solid outer phase of the particle is melted or liquefied, e.g., by exposing the solid phase to a threshold temperature or a melting temperature capable of melting the solid phase, thereby causing release of the species from the inner phase of the particle, i.e., the presence of any auxiliary stabilizing agent that may be present within the particle does not significantly alter the threshold or melting temperature, or other condition in which the particle releases the species, compared to a similar particle in which no auxiliary stabilizing agent is present.

In some embodiments, one or more fluidic droplets, or portions thereof, may be solidified. Any technique for solidifying a fluidic droplet such that at least a portion of the droplet solidifies can be used. For example, a fluidic droplet, or portion thereof, may be cooled to a temperature below the melting point or glass transition temperature of the droplet portion, a chemical reaction may be used that causes the fluid portion to solidify (for example, a polymerization reaction, a reaction between two fluids that produces a solid product, etc.), or the like. The solidification may be partial or complete, e.g., an entire phase of a fluidic droplet (e.g., an outer phase) may be solidified to form a solid, or only a portion of the phase (e.g., the outer phase) may be solidified to form a solid while other portions of the phase may remain liquid. As noted herein, the solid portion may be an amorphous or a crystalline solid, a semi-solid, or the like.

In one embodiment, the fluidic droplet, or a portion thereof, is solidified by reducing the temperature of the fluidic droplet to a temperature that causes at least one of the components of the fluidic droplet to reach a solid state or a glassy state. For example, a fluidic droplet (or portion thereof) may be solidified by cooling the fluidic droplet to a temperature that is below the melting point or glass transition temperature of a component or portion of the fluidic droplet, thereby causing the component or portion to become solid. As non-limiting examples, the fluidic droplet may be formed at an elevated temperature (e.g., above room temperature, about 25° C.), then cooled (e.g., to room temperature or to a temperature below room temperature); or the fluidic droplet may be formed at room temperature, then cooled to a temperature below room temperature, or the like.

In some cases, an outer fluid portion surrounding an inner droplet, or other fluid portion, may be hardened, such as by solidifying or gelling the outer fluid to form a shell surrounding the inner fluid. In this way, capsules or particles can be formed, and in some cases with consistently and/or monodisperse inner droplets, and/or in some cases with consistent and/or monodisperse outer shells. In some embodiments, monodisperse particles may be formed. In some embodiments, this can be accomplished by a phase change in the outer fluid. In various embodiments, the entire droplet (including any or all inner fluids) may be solidified, or only a portion of the droplet may be solidified (e.g., a portion of an outer fluid may be solidified). For example, in some embodiments, the outer phase of a multiple emulsion droplet may have a different melting temperature than the inner phase, so that upon exposure of the multiple emulsion droplet to a certain temperature, the outer phase of the multiple emulsion droplet may solidify while the inner phase does not solidify, and/or the inner phase may solidify while the outer phase does not solidify.

A phase change in a fluidic phase in a multiple emulsion droplet can be initiated by a temperature change, for instance, and in some cases, the phase change is reversible. For example, a wax or a gel may be used as a fluid at a temperature which maintains the wax or gel as a fluid. Upon cooling, the wax or gel can form a solid phase, e.g., resulting in a capsule or a particle. In some cases, the solid phase may exhibit semisolid or quasi-solid properties, e.g., exhibiting a viscosity and/or a rigidity intermediate between that of a solid and a liquid. The solid phase may also be amorphous or crystalline. Thus, for example, a multiple emulsion droplet may be formed using such a wax or gel under conditions in which the wax or gel is a liquid (e.g., by forming the multiple emulsion droplet at a temperature greater than the melting point of the wax or gel), then the multiple emulsion droplet allowed to cool to cause the wax or gel to at least partially solidify, e.g., such that at least part of the wax or gel becomes solid. For instance, if the wax or gel is formed as the outer phase of a multiple emulsion droplet, when the wax or gel is cooled to cause the wax or gel to at least partially solidify, a capsule or particle may be formed where the wax or gel encapsulates or surrounds an inner fluid. Non-limiting examples of waxes or gels include poly(N-isopropylacrylamide), fatty glycerides, paraffin oil, nonadecane, eicosane, or the like.

In some cases, a multiple emulsion droplet may comprise a fluidic portion having a sol state and a gel state, such that the conversion of the fluidic portion from the sol state into a gel state causes the portion to solidify. The conversion of a sol state of the fluidic portion into a gel state may be accomplished through any technique known to those of ordinary skill in the art, for instance, by cooling the fluidic portion, by initiating a polymeric reaction within the fluidic portion, etc. For example, agarose is used, a fluidic droplet, such as a multiple emulsion droplet, containing agarose may be produced at a temperature above the gelling temperature of agarose, then the droplet subsequently cooled, causing the agarose to enter a gel state. As another example, if acrylamide is used (e.g., dissolved within a fluidic droplet, such as a multiple emulsion droplet), the acrylamide may be polymerized (e.g., using APS (ammonium persulfate) and tetramethylethylenediamine) to produce a polymeric particle comprising polyacrylamide.

In another set of embodiments, a phase change can be initiated by a pressure change. For example, a multiple emulsion droplet may be formed at a first pressure where a phase of the droplet is liquid or fluid, for instance, where the phase is an outer fluid or an inner fluid. Decreasing or increasing the pressure to a second pressure may cause the phase to at least partially solidify. Non-limiting examples of such fluids include baroplastic polymers such as copolymers of polystyrene and poly(butyl acrylate) or poly(2-ethyl hexyl acrylate).

In another set of embodiments, the fluidic droplet, or portion thereof, may be solidified using a chemical reaction that causes solidification of the fluidic portion to occur. For example, two or more reactants added to a fluidic droplet may react to produce a solid product, thereby causing solidification of the portion to occur. As another example, a first reactant within the fluidic droplet may be reacted with a second reactant within the liquid surrounding the fluidic droplet to produce a solid, which may thus coat the fluidic droplet within a solid “shell” in some cases, thereby forming a core/shell particle or capsule having a solid shell or exterior, and a fluidic core or interior.

In yet another set of embodiments, a particle can be formed by polymerizing one or more phases in a multiple emulsion droplet, for example, an outer phase and/or an inner phase. Polymerization can be accomplished in a number of ways, including using a pre-polymer or a monomer that can be catalyzed, for example, chemically, through heat, or via electromagnetic radiation (e.g., ultraviolet radiation) to form a solid polymer shell. For example, a polymerization reaction may be initiated within a fluidic droplet or portion thereof, thereby causing the formation of a polymeric particle. For instance, the fluidic droplet may contain one or more monomer or oligomer precursors (e.g., dissolved and/or suspended within the fluidic droplet), which may polymerize to form a polymer that is solid. The polymerization reaction may occur spontaneously, or be initiated in some fashion, e.g., during formation of the fluidic droplet, or after the fluidic droplet has been formed. For instance, the polymerization reaction may be initiated by adding an initiator to the fluidic droplet, by applying light or other electromagnetic energy to the fluidic droplet (e.g., to initiate a photopolymerization reaction), or the like.

A non-limiting example of a solidification reaction is a polymerization reaction involving production of a nylon (e.g., a polyamide), for example, from a diacyl chloride and a diamine. Those of ordinary skill in the art will know of various suitable nylon-production techniques. For example, nylon-6,6 may be produced by reacting adipoyl chloride and 1,6-diaminohexane. For instance, a fluidic droplet, or portion thereof, may be solidified by reacting adipoyl chloride in the continuous phase with 1,6-diaminohexane within the fluidic droplet, which can react to form nylon-6,6 at the surface of the fluidic droplet. Depending on the reaction conditions, nylon-6,6 may be produced at the surface of the fluidic droplet (e.g., forming a particle having a solid exterior and a fluidic interior), or within the fluidic droplet (e.g., forming a solid particle).

Additionally, a polymer of a solidified particle can, in some embodiments, be degraded to return the phase to an essentially fluid state. For example, a polymer may be degradable hydrolytically, enzymatically, photolytically, etc. In some embodiments, the polymer may exhibit a phase change from a solid or “glassy” phase to a “rubbery” phase, and in some cases, an agent may be able to pass through the polymer when the polymer is in a rubbery phase but not when the polymer is in a solid phase. For example, the polymer may exhibit such a phase change upon being heated to at least its glass transition temperature.

In some embodiments, an agent or other contents of a particle may be released above a threshold temperature of the particle. The “threshold temperature” of a particle is the temperature above which the particle releases an agent or other content contained internally within the particle; below the threshold temperature, the particle is not able to release the agent or other content, or at least does not release a detectable amount of the agent within a relatively long period of time, e.g., at least a day. In some embodiments, the threshold temperature is a specific, well-defined temperature; for example, the temperature may be a melting temperature of a phase of the particle. In other embodiments, however, the threshold temperature may be more accurately described as a range or a gradient; for example, certain polymers may have a melting temperature or exhibit a transition (e.g., from a glassy phase to a rubbery phase) over a temperature range. As another example, the threshold temperature may be a glass transition temperature of a polymer.

As certain examples, release of an agent from a particle may occur by melting at least a portion of the particle. In some embodiments, the threshold temperature may be the melting temperature of one or more phases of the particle, for example, an outer phase or an outer phase. The threshold temperature may also be a degradation temperature (e.g., a temperature at which the solid phase material begins to degrade or decompose) or a glass transition temperature. In some embodiments, a particle may have a predetermined threshold temperature. In some embodiments, the threshold temperature (e.g., a melting temperature, a glass transition temperature, a degradation temperature, etc.) of a particle may be at least 0° C., at least 10° C., at least 20° C., at least 30° C., at least 40° C., at least 50° C., at least 60° C., at least 70° C., at least 80° C., or even higher. In some embodiments, the threshold temperature (e.g., a melting temperature, a glass transition temperature, a degradation temperature, etc.) of the particle may be between 0° C. and 20° C., between 15° C. and 35° C., between 30° C. and 50° C., or between 45° C. and 65° C. It should be understood that temperatures outside these ranges may be used as well, in certain embodiments. In addition, the pressure may be controlled instead of and/or in addition to the temperature, e.g., to cause release of an agent. For instance, one of ordinary skill in the art would recognize that the melting temperature of a solid phase (e.g., the outer phase of a particle) may be affected by the pressure.

A particle may be able to release a species upon exposure of the particle to a temperature greater than or equal to a threshold temperature, e.g., of a phase of the particle, for example, an outer phase. The threshold temperature may be, for instance, a melting temperature, a degradation temperature, a glass transition temperature, etc. In some cases, release of the species may be relatively rapid. For example, in some embodiments, a particle may be able to release at least 50% of the species contained within the particle within 1 minute of exposure of the particle to a threshold temperature, within 5 minutes of exposure of the particle to a threshold temperature, within 10 minutes of exposure of the particle to a threshold temperature, within 30 minutes of exposure of the particle to a threshold temperature, or within 1 hour of exposure of the particle to a threshold temperature.

However, in some embodiments, the particle may be able to retain the species for a much longer period of time, after exposure of the particle to temperatures greater than the threshold temperature. For example, in some embodiments, less than 5% of a species may be released by a particle after a period of at least 1 week, at least 2 weeks, at least 1 month (4 weeks), at least 6 months (26 weeks), or at least one year, even while the particle is continually exposed to temperatures above the threshold temperature.

The outer phase of a particle (or any other phase of the particle in which the phase may be controlled as discussed herein) may be formed from any suitable material. In some embodiments, the outer phase may be selected to be essentially immiscible with an inner phase or other phase of the particle, for example when the outer phase (or other phase) is in a liquid state. Examples of suitable materials in which the phase may be controlled include, but are not limited to, oils such as glyceride (melting point of 33° C. to 35° C.), paraffin oil (melting point of 42° C. to 45° C.), nonadecane (melting point of 32° C.), and eicosane (melting point of 37° C.). The melting point can be determined using any suitable technique known to those of ordinary skill in the art, for example, a Thiele tube, a Fisher-Johns apparatus, a Gallenkamp melting point apparatus, or the like (it should also be noted that “melting point” (the solid-to-liquid transition temperature) is typically determined, rather than the closely related “freezing point” (the liquid-to-solid transition temperature), due to the ability of some substances to supercool).

Typically, a substance has a single melting temperature, where the substance transitions from a solid phase to a liquid phase. However, it should be understood that, in some cases, it may not be possible to rigorously define a single melting temperature for certain types of materials, i.e., the material may exhibit a range of melting temperatures, such as noted in the examples above. In some embodiments, a mixture of components may be used as the outer phase or other phase. Those of ordinary skill in the art will be able to predict such melting temperatures, and can use such information to select one or more materials to have a predetermined melting point. For example, a mixture of two or more components with different melting temperatures may be used, and in some embodiments, the melting temperature of the mixture may be predetermined by controlling the ratio of the at least two components together within the mixture. Those of ordinary skill in the art will be able to select a ratio of two or more components within a mixture. In some cases, the melting temperature or threshold temperature of the mixture of components may be controlled by controlling the ratio of the components forming the mixture, e.g., such that a certain predetermined melting temperature or threshold temperature of the mixture is achieved, for example, using routine techniques and calculations known to those of ordinary skill in the art. For example, a particle may contain a mixture of glyceride and paraffin, and the exact melting temperature or threshold temperature may be controlled by controlling the weight or mass ratio of glyceride to paraffin within the mixture.

The inner phase, or other phase which is not subjected to a phase change, may be any suitable material, and may be solid, liquid, gas, etc., in various embodiments In some embodiments, for example, the inner phase is an aqueous solution. The inner phase (or other phase) may also comprise or contain one or more species, as discussed herein. In some embodiments, the inner phase may comprise additional components. For example, the inner phase (or other phase) may comprise a component that increases the viscosity of the inner phase, such as glycerol.

As discussed above, particles may be stabilized, in some embodiments, due to the at least partially solidified portions of the particles, for example, an outer phase of the particle. Advantageously, this method can be used, in some embodiments, to contain or encapsulate a species such as an amphiphilic compound. Generally, amphiphilic compounds can be difficult to encapsulate using prior art techniques, for example, prior art techniques for creating multiple emulsion droplets. Without wishing to be bound by any theory, an amphiphilic compound may, in some instances, disrupt the oil-water interface of droplets within an emulsion, thereby significantly reducing the half-life of the droplets within the emulsion and/or by preventing or inhibiting formation of a multiple emulsion droplet. However, by solidifying at least a portion of a droplet containing an amphiphilic compound to form a particle, as discussed herein, a particle can contain an amphiphilic compound. The particle thus formed may be stable, as discussed herein, and in some cases the particle containing the amphiphilic compound may be indefinitely stable. In some embodiments, the particle can also be caused to release the amphiphilic compound when desired, for instance, by exposing the particle to a threshold temperature that is able to cause at least a portion of the particle to release the amphiphilic compound; for example, a solid outer portion of the particle may be melted or liquefied to allow release of the amphiphilic compound from the particle.

Fields in which colloidal systems and other systems as discussed herein may prove useful include, for example, food, beverage, health and beauty aids, paints and coatings, household products (e.g., detergent), and drugs and drug delivery. For instance, a precise quantity of a drug, pharmaceutical, or other agent can be contained within an emulsion, or in some instances, cells can be contained within a droplet, and the cells can be stored and/or delivered. Other species or agents that can be stored and/or delivered include, for example, biochemical species such as nucleic acids such as siRNA, RNAi and DNA, proteins, peptides, or enzymes, or the like. Additional species or agents that can be incorporated within an emulsion of the invention include, but are not limited to, nanoparticles, quantum dots, fragrances, proteins, indicators, dyes, fluorescent species, chemicals, amphiphilic compounds, detergents, drugs, or the like. Further additional species or agents that can be incorporated within an emulsion of the invention include, but are not limited to, pesticides, such as herbicides, fungicides, insecticides, growth regulators, and microbicides. An emulsion can also serve as a reaction vessel in certain cases, such as for controlling chemical reactions, or for in vitro transcription and translation, e.g., for directed evolution technology.

Thus, in certain embodiments of the invention, the fluidic droplets (or a portion thereof) may contain additional species, for example, other chemical, biochemical, or biological entities (e.g., dissolved or suspended in the fluid), cells, particles, gases, molecules, pharmaceutical agents, drugs, DNA, RNA, proteins, fragrance, reactive agents, biocides, fungicides, preservatives, chemicals, amphiphilic compounds, or the like. In some embodiments, the fluidic droplets (or a portion thereof) may contain additional entities or species, for example, pesticides, such as herbicides, fungicides, insecticides, growth regulators, and microbiocides. Cells, for example, can be suspended in a fluid emulsion. Thus, the species may be any substance that can be contained in any portion of an emulsion. The species may be present in any fluidic droplet, for example, within an inner droplet, within an outer droplet, etc. For instance, one or more cells and/or one or more cell types can be contained in a droplet.

The term “determining,” as used herein, generally refers to the analysis or measurement of a species, for example, quantitatively or qualitatively, and/or the detection of the presence or absence of the species. “Determining” may also refer to the analysis or measurement of an interaction between two or more species, for example, quantitatively or qualitatively, or by detecting the presence or absence of the interaction. Examples of suitable techniques include, but are not limited to, spectroscopy such as infrared, absorption, fluorescence, UV/visible, FTIR (“Fourier Transform Infrared Spectroscopy”), or Raman; gravimetric techniques; ellipsometry; piezoelectric measurements; immunoassays; electrochemical measurements; optical measurements such as optical density measurements; circular dichroism; light scattering measurements such as quasielectric light scattering; polarimetry; refractometry; or turbidity measurements.

In some embodiments, a species may be encapsulated with relatively high efficiency. For example, a multiple emulsion droplet may be formed where a species is encapsulated within the droplet. In some cases, at least about 50%, at least about 60%, at least about 70%, at least about 80%, at least about 90%, at least about 95%, at least about 98% of a species may be encapsulated, or essentially all of the species may be encapsulated within the multiple emulsion droplet, i.e., during the process used to form the multiple emulsion droplet, at least about 50%, at least about 60%, etc. of a species that is introduced during the formation process is actually contained within multiple emulsion droplets after the droplets are formed.

In one set of embodiments of the present invention, a double emulsion droplet or other multiple emulsion droplet is produced, i.e., a carrying fluid, containing an outer fluidic droplet, which in turn contains an inner fluidic droplet therein. In some cases, the carrying fluid and the inner fluid may be the same. These fluids are often of varying miscibilities due to differences in hydrophobicity. For example, the first fluid may be water soluble, the second fluid oil soluble, and the carrying fluid water soluble. This arrangement is often referred to as a w/o/w multiple emulsion (“water/oil/water”). Another multiple emulsion may include a first fluid that is oil soluble, a second fluid that is water soluble, and a carrying fluid that is oil soluble. This type of multiple emulsion is often referred to as an o/w/o multiple emulsion (“oil/water/oil”). It should be noted that the term “oil” in the above terminology merely refers to a fluid that is generally more hydrophobic and not miscible in water, as is known in the art. Thus, the oil may be a hydrocarbon in some embodiments, but in other embodiments, the oil may comprise other hydrophobic fluids. It should also be understood that the water need not be pure; it may be an aqueous solution, for example, a buffer solution, a solution containing a dissolved salt, or the like.

More specifically, as used herein, two fluids are immiscible, or not miscible, with each other when one is not soluble in the other to a level of at least 10% by weight at the temperature and under the conditions at which the emulsion is produced. For instance, two fluids may be selected to be immiscible within the time frame of the formation of the fluidic droplets. In some embodiments, the fluids used to form a multiple emulsion may the same, or different. For example, in some cases, two or more fluids may be used to create a multiple emulsion, and in certain instances, some or all of these fluids may be immiscible. In some embodiments, two fluids used to form a multiple emulsion are compatible, or miscible, while a middle fluid contained between the two fluids is incompatible or immiscible with these two fluids. In other embodiments, however, all three fluids may be mutually immiscible, and in certain cases, all of the fluids do not all necessarily have to be water soluble.

More than two fluids may be used in other embodiments of the invention. Accordingly, certain embodiments of the present invention are generally directed to multiple emulsions, which includes larger fluidic droplets that contain one or more smaller droplets therein which, in some cases, can contain even smaller droplets therein, etc. Any number of nested fluids can be produced, and accordingly, additional third, fourth, fifth, sixth, etc. fluids may be added in some embodiments of the invention to produce increasingly complex droplets within droplets. It should be understood that not all of these fluids necessarily need to be distinguishable; for example, a quadruple emulsion containing oil/water/oil/water or water/oil/water/oil may be prepared, where the two oil phases have the same composition and/or the two water phases have the same composition.

A “droplet,” as used herein, is an isolated portion of a first fluid that is surrounded by a second fluid. It is to be noted that a droplet is not necessarily spherical, but may assume other shapes as well, for example, depending on the external environment. In one embodiment, the droplet has a minimum cross-sectional dimension that is substantially equal to the largest dimension of the channel perpendicular to fluid flow in which the droplet is located. In some cases, the droplets will have a homogenous distribution of diameters, i.e., the droplets may have a distribution of diameters such that no more than about 10%, about 5%, about 3%, about 1%, about 0.03%, or about 0.01% of the droplets have an average diameter greater than about 10%, about 5%, about 3%, about 1%, about 0.03%, or about 0.01% of the average diameter of the droplets, and correspondingly, droplets within the outlet channel may have the same, or similar, distribution of diameters. Techniques for producing such a homogenous distribution of diameters are also disclosed in International Patent Application No. PCT/US2004/010903, filed Apr. 9, 2004, entitled “Formation and Control of Fluidic Species,” by Link, et al., published as WO 2004/091763 on Oct. 28, 2004, incorporated herein by reference, and in other references as described herein.

The fluids may be chosen such that the droplets remain discrete, relative to their surroundings. As non-limiting examples, a fluidic droplet may be created having a carrying fluid, containing a first fluidic droplet, containing a second fluidic droplet. In some cases, the carrying fluid and the second fluid may be identical or substantially identical; however, in other cases, the carrying fluid, the first fluid, and the second fluid may be chosen to be essentially mutually immiscible. One non-limiting example of a system involving three essentially mutually immiscible fluids is a silicone oil, a mineral oil, and an aqueous solution (i.e., water, or water containing one or more other species that are dissolved and/or suspended therein, for example, a salt solution, a saline solution, a suspension of water containing particles or cells, or the like). Another example of a system is a silicone oil, a fluorocarbon oil, and an aqueous solution. Yet another example of a system is a hydrocarbon oil (e.g., hexadecane), a fluorocarbon oil, and an aqueous solution. Non-limiting examples of suitable fluorocarbon oils include HFE 7500, octadecafluorodecahydronaphthalene:

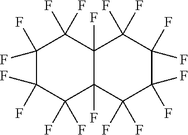

or 1-(1,2,2,3,3,4,4,5,5,6,6-undecafluorocyclohexyl)ethanol:

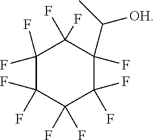

In the descriptions herein, multiple emulsions are often described with reference to a three phase system, i.e., having an outer or carrying fluid, a first fluid, and a second fluid. However, it should be noted that this is by way of example only, and that in other systems, additional fluids may be present within the multiple emulsion droplet. Accordingly, it should be understood that the descriptions such as the carrying fluid, first fluid, and second fluid are by way of ease of presentation, and that the descriptions herein are readily extendable to systems involving additional fluids, e.g., quadruple emulsions, quintuple emulsions, sextuple emulsions, septuple emulsions, etc.

As fluid viscosity can affect droplet formation, in some cases the viscosity of any of the fluids in the fluidic droplets may be adjusted by adding or removing components, such as diluents, that can aid in adjusting viscosity. For example, in some embodiments, the viscosity of the first fluid and the second fluid are equal or substantially equal. This may aid in, for example, an equivalent frequency or rate of droplet formation in the first and second fluids. In other embodiments, the viscosity of the first fluid may be equal or substantially equal to the viscosity of the second fluid, and/or the viscosity of the first fluid may be equal or substantially equal to the viscosity of the carrying fluid. In yet another embodiment, the carrying fluid may exhibit a viscosity that is substantially different from the first fluid. A substantial difference in viscosity means that the difference in viscosity between the two fluids can be measured on a statistically significant basis. Other distributions of fluid viscosities within the droplets are also possible. For example, the second fluid may have a viscosity greater than or less than the viscosity of the first fluid (i.e., the viscosities of the two fluids may be substantially different), the first fluid may have a viscosity that is greater than or less than the viscosity of the carrying fluid, etc. It should also be noted that, in higher-order droplets, e.g., containing four, five, six, or more fluids, the viscosities may also be independently selected as desired, depending on the particular application.

Using the methods and devices described herein, in some embodiments, an emulsion having a consistent size and/or number of droplets or particles can be produced, and/or a consistent ratio of size and/or number of outer phase droplets or portions to inner phase droplets or portions (or other such ratios) can be produced for cases involving multiple emulsions or particles formed therefrom. For example, in some cases, a single droplet within an outer droplet or particle of predictable size can be used to provide a specific quantity of a drug. In addition, combinations of compounds or drugs may be stored, transported, or delivered in a droplet or particle. For instance, hydrophobic, hydrophilic, and/or amphiphilic species can be delivered in a single, multiple emulsion droplet or particle formed therefrom, as the droplet or particle can include both hydrophilic and hydrophobic portions and can be at least partially solidified to stabilize an interface therein. The amount and concentration of each of these portions can be consistently controlled according to certain embodiments of the invention, which can provide for a predictable and consistent ratio of two or more species in a multiple emulsion droplet or particle.

Thus, in various embodiments, the droplets or particles formed therefrom may be of substantially the same shape and/or size (i.e., “monodisperse”), or of different shapes and/or sizes, depending on the particular application. As used herein, the term “fluid” generally refers to a substance that tends to flow and to conform to the outline of its container, i.e., a liquid, a gas, a viscoelastic fluid, etc. However, as discussed elsewhere herein, one of ordinary skill in the art would recognize that a fluid may undergo a phase change (e.g., from liquid to solid). Typically, fluids are materials that are unable to withstand a static shear stress, and when a shear stress is applied, the fluid experiences a continuing and permanent distortion. The fluid may have any suitable viscosity that permits flow. If two or more fluids are present, each fluid may be independently selected among essentially any fluids (liquids, gases, and the like) by those of ordinary skill in the art, by considering the relationship between the fluids. In some cases, the droplets may be contained within a carrier fluid, e.g., a liquid. It should be noted, however, that the present invention is not limited to only multiple emulsions. In some embodiments, single emulsions can also be produced.

In one set of embodiments, a monodisperse emulsion may be produced, e.g., as noted above. The shape and/or size of the fluidic droplets, or particles produced therefore can be determined, for example, by measuring the average diameter or other characteristic dimension of the droplets or particles. As discussed above, the droplets may be at least partially solidified to form solid particles. The “average diameter” of a plurality or series of droplets or particles is the arithmetic average of the average diameters of each of the droplets or particles. Those of ordinary skill in the art will be able to determine the average diameter (or other characteristic dimension) of a plurality or series of droplets or particles, for example, using laser light scattering, microscopic examination, or other known techniques. The average diameter of a single droplet or particle, in a non-spherical colloidal particle, is the diameter of a perfect sphere having the same volume as the droplet or particle. The average diameter of a droplet or particle (and/or of a plurality or series of droplets or particles) may be, for example, less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers in some cases. The average diameter may also be at least about 1 micrometer, at least about 2 micrometers, at least about 3 micrometers, at least about 5 micrometers, at least about 10 micrometers, at least about 15 micrometers, or at least about 20 micrometers in certain cases.

The rate of production of droplets (or particles) may be, in some embodiments, between approximately 100 Hz and 5,000 Hz. In some cases, the rate of droplet production may be at least about 200 Hz, at least about 300 Hz, at least about 500 Hz, at least about 750 Hz, at least about 1,000 Hz, at least about 2,000 Hz, at least about 3,000 Hz, at least about 4,000 Hz, or at least about 5,000 Hz, etc. In addition, production of large quantities of droplets or particles can be facilitated by the parallel use of multiple devices in some instances. In some cases, relatively large numbers of devices may be used in parallel, for example at least about 10 devices, at least about 30 devices, at least about 50 devices, at least about 75 devices, at least about 100 devices, at least about 200 devices, at least about 300 devices, at least about 500 devices, at least about 750 devices, or at least about 1,000 devices or more may be operated in parallel. The devices may comprise different channels, orifices, microfluidics, etc. In some cases, an array of such devices may be formed by stacking the devices horizontally and/or vertically. The devices may be commonly controlled, or separately controlled, and can be provided with common or separate sources of fluids, depending on the application. Examples of such systems are also described in U.S. Provisional Patent Application Ser. No. 61/160,184, filed Mar. 13, 2009, entitled “Scale-up of Microfluidic Devices,” by Romanowsky, et al., incorporated herein by reference.

In certain aspects, double or multiple emulsions containing relatively thin layers of fluid may be formed, e.g., using techniques such as those discussed herein. In some instances, one or more fluids may be hardened, e.g., to produce particles.

In one set of embodiments, a fluid “shell” surrounding a droplet may be defined as being between two interfaces, a first interface between a first fluid and a carrying fluid, and a second interface between the first fluid and a second fluid. The interfaces may have an average distance of separation (determined as an average over the droplet) that is no more than about 1 mm, about 300 micrometers, about 100 micrometers, about 30 micrometers, about 10 micrometers, about 3 micrometers, about 1 micrometers, etc. In some cases, the interfaces may have an average distance of separation defined relative to the average dimension of the droplet. For instance, the average distance of separation may be less than about 30%, less than about 25%, less than about 20%, less than about 15%, less than about 10%, less than about 5%, less than about 3%, less than about 2%, or less than about 1% of the average dimension of the droplet.

Examples of fluid hardening techniques useful for forming hardened droplets and/or hardened streams of fluid include those discussed in detail below, as well as those disclosed in International Patent Application No. PCT/US2004/010903, filed Apr. 9, 2004, entitled “Formation and Control of Fluidic Species,” by Link, et al., published as WO 2004/091763 on Oct. 28, 2004; U.S. patent application Ser. No. 11/368,263, filed Mar. 3, 2006, entitled “Systems and Methods of Forming Particles,” by Garstecki, et al., published as U.S. Patent Application Publication No. 2007/0054119 on Mar. 8, 2007; or U.S. patent application Ser. No. 11/885,306, filed Aug. 29, 2007, entitled “Method and Apparatus for Forming Multiple Emulsions,” by Weitz, et al., published as U.S. Patent Application Publication No. 2009/0131543 on May 21, 2009, each incorporated herein by reference.

As discussed, in various aspects of the present invention, multiple emulsions are formed by flowing two, three, or more fluids through various conduits or channels. One or more (or all) of the channels may be microfluidic. “Microfluidic,” as used herein, refers to a device, apparatus or system including at least one fluid channel having a cross-sectional dimension of less than about 1 millimeter (mm), and in some cases, a ratio of length to largest cross-sectional dimension of at least 3:1. One or more channels of the system may be a capillary tube. In some cases, multiple channels are provided. The channels may be in the microfluidic size range and may have, for example, average inner diameters, or portions having an inner diameter, of less than about 1 millimeter, less than about 300 micrometers, less than about 100 micrometers, less than about 30 micrometers, less than about 10 micrometers, less than about 3 micrometers, or less than about 1 micrometer, thereby providing droplets having comparable average diameters. One or more of the channels may (but not necessarily), in cross section, have a height that is substantially the same as a width at the same point. In cross-section, the channels may be rectangular or substantially non-rectangular, such as circular or elliptical.

The microfluidic channels may be arranged in any suitable system. As discussed above, in some embodiments, the main channel may be relatively straight, but in other embodiments, a main channel may be curved, angled, bent, or have other shapes. In some embodiments, the microfluidic channels may be arranged in a two dimensional pattern, i.e., such that the positions of the microfluidic channels can be described in two dimensions such that no micro fluidic channels cross each other without the fluids therein coming into physical contact with each other, e.g., at an intersection. Of course, such channels, even though represented as a planar array of channels (i.e., in a quasi-two dimensional array of channels), are not truly two-dimensional, but have a length, width and height. In contrast, for instance, a “tube-within-a-tube” configuration would not be quasi-two dimensional, as there is at least one location in which the fluids within two microfluidic channels do not physically come into contact with each other, although they appear to do so in two dimensions.

A “channel,” as used herein, means a feature on or in an article (substrate) that at least partially directs flow of a fluid. The channel can have any cross-sectional shape (circular, oval, triangular, irregular, square or rectangular, or the like) and can be covered or uncovered. In embodiments where it is completely covered, at least one portion of the channel can have a cross-section that is completely enclosed, or the entire channel may be completely enclosed along its entire length with the exception of its inlet(s) and/or outlet(s). A channel may also have an aspect ratio (length to average cross sectional dimension) of at least 2:1, more typically at least 3:1, 5:1, 10:1, 15:1, 20:1, or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to fluid flow of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases the dimensions of the channel may be chosen such that fluid is able to freely flow through the article or substrate. The dimensions of the channel may also be chosen, for example, to allow a certain volumetric or linear flow rate of fluid in the channel. Of course, the number of channels and the shape of the channels can be varied by any method known to those of ordinary skill in the art. In some cases, more than one channel or capillary may be used. For example, two or more channels may be used, where they are positioned inside each other, positioned adjacent to each other, positioned to intersect with each other, etc.

Thus, in certain embodiments, the present invention is generally directed to methods of creating multiple emulsions, including double emulsions, triple emulsions, and other higher-order emulsions, and/or particles formed from such emulsions. In one set of embodiments, a fluid flows through a channel, and is surrounded by another fluid. In some cases, the two fluids may flow in a collinear fashion, e.g., without creating individual droplets. The two fluids may then be surrounded by yet another fluid, which may flow collinearly with the first two fluids in some embodiments, and/or cause the fluids to form discrete droplets within the channel. In some cases, streams of multiple collinear fluids may be formed, and/or caused to form triple or higher-order emulsions. In some cases, as discussed below, this may occur as a single process, e.g., the multiple emulsion is formed at substantially the same time from the various streams of collinear fluids. As discussed, in certain embodiments, one or more portions or phase of the multiple emulsion may be solidified, e.g., to produce particles such as those discussed herein.

In one set of embodiments, an inner fluid flows through a main channel, while an outer fluid flows into a first intersection through one or more side channels to the main channel, and a carrying fluid flows into a second intersection through one or more side channels. In some cases, the outer fluid, upon entry into the main channel, may surround the inner fluid without causing the inner fluid to form separate droplets. For instance, the inner fluid and the outer fluid may flow collinearly within the main channel. The outer fluid, in some cases, may surround the inner fluid, preventing the inner fluid from contacting the walls of the fluidic channel; for instance, the channel may widen upon entry of the outer fluid in some embodiments. In some cases, additional channels may bring additional fluids to the main channel without causing droplet formation to occur. In certain instances, a carrying fluid may be introduced into the main channel, surrounding the inner and outer fluids. In some cases, introduction of the carrying fluid may cause the fluids to form into separate droplets (e.g., of an inner fluid, surrounded by an outer fluid, which is in turn surrounded by a carrying fluid). The carrying fluid, in some embodiments, may prevent the inner and/or outer fluids from contacting the walls of fluidic channel; for instance, the channel may widen upon entry of the carrying fluid, or in some cases, carrying fluid may be added using more than one side channel and/or at more than one intersection.

In some cases, more than three fluids may be present. For example, there may be four, five, six, or more fluids flowing collinearly within a microfluidic channel, e.g., formed using techniques such as those described herein, and in some cases, repeatedly used, e.g., involving three, four, five, six, etc., or more intersections, multiple changes in hydrophilicity and/or average cross-sectional dimension, or the like. In some cases, some or all of these fluids may exhibit dripping or jetting behavior. For instance, multiple collinear streams of fluid may be formed within a microfluidic channel, and in some cases, one or more of the streams of fluid may exhibit dripping or jetting behavior. In some embodiments, the collinearly flowing fluids may be caused to form a multiple emulsion droplet, as discussed herein. In some cases, the multiple emulsion droplet may be formed in a single step, e.g., without creating single or double emulsion droplets prior to creating the multiple emulsion droplet.

In some embodiments, multiple emulsions such as those described herein may be prepared by controlling the hydrophilicity and/or hydrophobicity of the channels used to form the multiple emulsion, according to some (but not all) embodiments. In one set of embodiments, the hydrophilicity and/or hydrophobicity of the channels may be controlled by coating a sol-gel onto at least a portion of a channel. For instance, in one embodiment, relatively hydrophilic and relatively hydrophobic portions may be created by applying a sol-gel to the channel surfaces, which renders them relatively hydrophobic. The sol-gel may comprise an initiator, such as a photoinitiator. Portions (e.g., channels, and/or portions of channels) may be rendered relatively hydrophilic by filling the channels with a solution containing a hydrophilic moiety (for example, acrylic acid), and exposing the portions to a suitable trigger for the initiator (for example, light or ultraviolet light in the case of a photoinitiator). For example, the portions may be exposed by using a mask to shield portions in which no reaction is desired, by directed a focused beam of light or heat onto the portions in which reaction is desired, or the like. In the exposed portions, the initiator may cause the reaction (e.g., polymerization) of the hydrophilic moiety to the sol-gel, thereby rendering those portions relatively hydrophilic (for instance, by causing poly(acrylic acid) to become grafted onto the surface of the sol-gel coating in the above example).

As is known to those of ordinary skill in the art, a sol-gel is a material that can be in a sol or a gel state, and typically includes polymers. The gel state typically contains a polymeric network containing a liquid phase, and can be produced from the sol state by removing solvent from the sol, e.g., via drying or heating techniques. In some cases, as discussed below, the sol may be pretreated before being used, for instance, by causing some polymerization to occur within the sol.

In some embodiments, the sol-gel coating may be chosen to have certain properties, for example, having a certain hydrophobicity. The properties of the coating may be controlled by controlling the composition of the sol-gel (for example, by using certain materials or polymers within the sol-gel), and/or by modifying the coating, for instance, by exposing the coating to a polymerization reaction to react a polymer to the sol-gel coating, as discussed below.

For example, the sol-gel coating may be made more hydrophobic by incorporating a hydrophobic polymer in the sol-gel. For instance, the sol-gel may contain one or more silanes, for example, a fluorosilane (i.e., a silane containing at least one fluorine atom) such as heptadecafluorosilane, or other silanes such as methyltriethoxy silane (MTES) or a silane containing one or more lipid chains, such as octadecylsilane or other CH₃(CH₂)ₙ-silanes, where n can be any suitable integer. For instance, n may be greater than 1, 5, or 10, and less than about 20, 25, or 30. The silanes may also optionally include other groups, such as alkoxide groups, for instance, octadecyltrimethoxysilane. In general, most silanes can be used in the sol-gel, with the particular silane being chosen on the basis of desired properties such as hydrophobicity. Other silanes (e.g., having shorter or longer chain lengths) may also be chosen in other embodiments of the invention, depending on factors such as the relative hydrophobicity or hydrophilicity desired. In some cases, the silanes may contain other groups, for example, groups such as amines, which would make the sol-gel more hydrophilic. Non-limiting examples include diamine silane, triamine silane, or N-[3-(trimethoxysilyl)propyl] ethylene diamine silane. The silanes may be reacted to form oligomers or polymers within the sol-gel, and the degree of polymerization (e.g., the lengths of the oligomers or polymers) may be controlled by controlling the reaction conditions, for example by controlling the temperature, amount of acid present, or the like. In some cases, more than one silane may be present in the sol-gel. For instance, the sol-gel may include fluorosilanes to cause the resulting sol-gel to exhibit greater hydrophobicity, and other silanes (or other compounds) that facilitate the production of polymers. In some cases, materials able to produce SiO₂ compounds to facilitate polymerization may be present, for example, TEOS (tetraethyl orthosilicate).

It should be understood that the sol-gel is not limited to containing only silanes, and other materials may be present in addition to, or in place of, the silanes. For instance, the coating may include one or more metal oxides, such as SiO₂, vanadia (V₂O₅), titania (TiO₂), and/or alumina (Al₂O₃).

In some instances, the microfluidic channel is present in a material suitable to receive the sol-gel, for example, glass, metal oxides, or polymers such as polydimethylsiloxane (PDMS) and other siloxane polymers. For example, in some cases, the microfluidic channel may be one in which contains silicon atoms, and in certain instances, the microfluidic channel may be chosen such that it contains silanol (Si—OH) groups, or can be modified to have silanol groups. For instance, the microfluidic channel may be exposed to an oxygen plasma, an oxidant, or a strong acid cause the formation of silanol groups on the microfluidic channel.

The sol-gel may be present as a coating on the microfluidic channel, and the coating may have any suitable thickness. For instance, the coating may have a thickness of no more than about 100 micrometers, no more than about 30 micrometers, no more than about 10 micrometers, no more than about 3 micrometers, or no more than about 1 micrometer. Thicker coatings may be desirable in some cases, for instance, in applications in which higher chemical resistance is desired. However, thinner coatings may be desirable in other applications, for instance, within relatively small microfluidic channels.

In one set of embodiments, the hydrophobicity of the sol-gel coating can be controlled, for instance, such that a first portion of the sol-gel coating is relatively hydrophobic, and a second portion of the sol-gel coating is relatively hydrophilic. The hydrophobicity of the coating can be determined using techniques known to those of ordinary skill in the art, for example, using contact angle measurements such as those discussed herein. For instance, in some cases, a first portion of a microfluidic channel may have a hydrophobicity that favors an organic solvent to water, while a second portion may have a hydrophobicity that favors water to the organic solvent.

The hydrophobicity of the sol-gel coating can be modified, for instance, by exposing at least a portion of the sol-gel coating to a polymerization reaction to react a polymer to the sol-gel coating. The polymer reacted to the sol-gel coating may be any suitable polymer, and may be chosen to have certain hydrophobicity properties. For instance, the polymer may be chosen to be more hydrophobic or more hydrophilic than the microfluidic channel and/or the sol-gel coating. As an example, a hydrophilic polymer that could be used is poly(acrylic acid).

The polymer may be added to the sol-gel coating by supplying the polymer in monomeric (or oligomeric) form to the sol-gel coating (e.g., in solution), and causing a polymerization reaction to occur between the monomer and the sol-gel. For instance, free radical polymerization may be used to cause bonding of the polymer to the sol-gel coating. In some embodiments, a reaction such as free radical polymerization may be initiated by exposing the reactants to heat and/or light, such as ultraviolet (UV) light, optionally in the presence of a photoinitiator able to produce free radicals (e.g., via molecular cleavage) upon exposure to light. Those of ordinary skill in the art will be aware of many such photoinitiators, many of which are commercially available, such as Irgacur 2959 (Ciba Specialty Chemicals) or 2-hydroxy-4-(3-triethoxysilylpropoxy)-diphenylketone (SIH6200.0, ABCR GmbH & Co. KG).

The photoinitiator may be included with the polymer added to the sol-gel coating, or in some cases, the photoinitiator may be present within the sol-gel coating. For instance, a photoinitiator may be contained within the sol-gel coating, and activated upon exposure to light. The photoinitiator may also be conjugated or bonded to a component of the sol-gel coating, for example, to a silane. As an example, a photoinitiator such as Irgacur 2959 may be conjugated to a silane-isocyanate via a urethane bond, where a primary alcohol on the photoinitiator may participate in nucleophilic addition with the isocyanate group, which may produce a urethane bond.

It should be noted that only a portion of the sol-gel coating may be reacted with a polymer, in some embodiments of the invention. For instance, the monomer and/or the photoinitiator may be exposed to only a portion of the microfluidic channel, or the polymerization reaction may be initiated in only a portion of the microfluidic channel. As a particular example, a portion of the micro fluidic channel may be exposed to light, while other portions are prevented from being exposed to light, for instance, by the use of masks or filters, or by using a focused beam of light. Accordingly, different portions of the microfluidic channel may exhibit different hydrophobicities, as polymerization does not occur everywhere on the microfluidic channel. As another example, the microfluidic channel may be exposed to UV light by projecting a de-magnified image of an exposure pattern onto the microfluidic channel. In some cases, small resolutions (e.g., 1 micrometer, or less) may be achieved by projection techniques.

Another aspect of the present invention is generally directed at systems and methods for coating such a sol-gel onto at least a portion of a microfluidic channel. In one set of embodiments, a microfluidic channel is exposed to a sol, which is then treated to form a sol-gel coating. In some cases, the sol can also be pretreated to cause partial polymerization to occur. Extra sol-gel coating may optionally be removed from the microfluidic channel. In some cases, as discussed, a portion of the coating may be treated to alter its hydrophobicity (or other properties), for instance, by exposing the coating to a solution containing a monomer and/or an oligomer, and causing polymerization of the monomer and/or oligomer to occur with the coating.

The sol may be contained within a solvent, which can also contain other compounds such as photoinitiators including those described above. In some cases, the sol may also comprise one or more silane compounds. The sol may be treated to form a gel using any suitable technique, for example, by removing the solvent using chemical or physical techniques, such as heat. For instance, the sol may be exposed to a temperature of at least about 150° C., at least about 200° C., or at least about 250° C., which may be used to drive off or vaporize at least some of the solvent. As a specific example, the sol may be exposed to a hotplate set to reach a temperature of at least about 200° C. or at least about 250° C., and exposure of the sol to the hotplate may cause at least some of the solvent to be driven off or vaporized. In some cases, however, the sol-gel reaction may proceed even in the absence of heat, e.g., at room temperature. Thus, for instance, the sol may be left alone for a while (e.g., about an hour, about a day, etc.), and/or air or other gases may be passed over the sol, to allow the sol-gel reaction to proceed.

In some cases, any ungelled sol that is still present may be removed from the microfluidic channel. The ungelled sol may be actively removed, e.g., physically, by the application of pressure or the addition of a compound to the microfluidic channel, etc., or the ungelled sol may be removed passively in some cases. For instance, in some embodiments, a sol present within a microfluidic channel may be heated to vaporize solvent, which builds up in a gaseous state within the microfluidic channels, thereby increasing pressure within the microfluidic channels. The pressure, in some cases, may be enough to cause at least some of the ungelled sol to be removed or “blown” out of the microfluidic channels.

In certain embodiments, the sol is pretreated to cause partial polymerization to occur, prior to exposure to the microfluidic channel. For instance, the sol may be treated such that partial polymerization occurs within the sol. The sol may be treated, for example, by exposing the sol to an acid or temperatures that are sufficient to cause at least some gellation to occur. In some cases, the temperature may be less than the temperature the sol will be exposed to when added to the microfluidic channel. Some polymerization of the sol may occur, but the polymerization may be stopped before reaching completion, for instance, by reducing the temperature. Thus, within the sol, some oligomers may form (which may not necessarily be well-characterized in terms of length), although full polymerization has not yet occurred. The partially treated sol may then be added to the microfluidic channel, as discussed above.

In certain embodiments, a portion of the coating may be treated to alter its hydrophobicity (or other properties) after the coating has been introduced to the microfluidic channel. In some cases, the coating is exposed to a solution containing a monomer and/or an oligomer, which is then polymerized to bond to the coating, as discussed above. For instance, a portion of the coating may be exposed to heat or to light such as ultraviolet right, which may be used to initiate a free radical polymerization reaction to cause polymerization to occur. Optionally, a photoinitiator may be present, e.g., within the sol-gel coating, to facilitate this reaction.

Additional details of such coatings and other systems may be seen in U.S. Provisional Patent Application Ser. No. 61/040,442, filed Mar. 28, 2008, entitled “Surfaces, Including Microfluidic Channels, With Controlled Wetting Properties,” by Abate, et al.; and International Patent Application Serial No. PCT/US2009/000850, filed Feb. 11, 2009, entitled “Surfaces, Including Microfluidic Channels, With Controlled Wetting Properties,” by Abate, et al., each incorporated herein by reference.

A variety of materials and methods, according to certain aspects of the invention, can be used to form systems (such as those described above) able to produce the multiple droplets described herein. In some cases, the various materials selected lend themselves to various methods. For example, various components of the invention can be formed from solid materials, in which the channels can be formed via micromachining, film deposition processes such as spin coating and chemical vapor deposition, laser fabrication, photolithographic techniques, etching methods including wet chemical or plasma processes, and the like. See, for example, Scientific American, 248:44-55, 1983 (Angell, et al). In one embodiment, at least a portion of the fluidic system is formed of silicon by etching features in a silicon chip. Technologies for precise and efficient fabrication of various fluidic systems and devices of the invention from silicon are known. In another embodiment, various components of the systems and devices of the invention can be formed of a polymer, for example, an elastomeric polymer such as polydimethylsiloxane (“PDMS”), polytetrafluoroethylene (“PTFE” or Teflon®), or the like.

Different components can be fabricated of different materials. For example, a base portion including a bottom wall and side walls can be fabricated from an opaque material such as silicon or PDMS, and a top portion can be fabricated from a transparent or at least partially transparent material, such as glass or a transparent polymer, for observation and/or control of the fluidic process. Components can be coated so as to expose a desired chemical functionality to fluids that contact interior channel walls, where the base supporting material does not have a precise, desired functionality. For example, components can be fabricated as illustrated, with interior channel walls coated with another material. Material used to fabricate various components of the systems and devices of the invention, e.g., materials used to coat interior walls of fluid channels, may desirably be selected from among those materials that will not adversely affect or be affected by fluid flowing through the fluidic system, e.g., material(s) that is chemically inert in the presence of fluids to be used within the device. A non-limiting example of such a coating was previously discussed.

In one embodiment, various components of the invention are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, or mixture of such polymers heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, etc.

Silicone polymers are preferred in one set of embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of the microfluidic structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric, and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures of the invention from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, components can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy, et al.), incorporated herein by reference.

In some embodiments, certain microfluidic structures of the invention (or interior, fluid-contacting surfaces) may be formed from certain oxidized silicone polymers. Such surfaces may be more hydrophilic than the surface of an elastomeric polymer. Such hydrophilic channel surfaces can thus be more easily filled and wetted with aqueous solutions.

In one embodiment, a bottom wall of a microfluidic device of the invention is formed of a material different from one or more side walls or a top wall, or other components. For example, the interior surface of a bottom wall can comprise the surface of a silicon wafer or microchip, or other substrate. Other components can, as described above, be sealed to such alternative substrates. Where it is desired to seal a component comprising a silicone polymer (e.g. PDMS) to a substrate (bottom wall) of different material, the substrate may be selected from the group of materials to which oxidized silicone polymer is able to irreversibly seal (e.g., glass, silicon, silicon oxide, quartz, silicon nitride, polyethylene, polystyrene, epoxy polymers, and glassy carbon surfaces which have been oxidized). Alternatively, other sealing techniques can be used, as would be apparent to those of ordinary skill in the art, including, but not limited to, the use of separate adhesives, bonding, solvent bonding, ultrasonic welding, etc.

The following applications are each incorporated herein by reference: U.S. patent application Ser. No. 08/131,841, filed Oct. 4, 1993, entitled “Formation of Microstamped Patterns on Surfaces and Derivative Articles,” by Kumar, et al., now U.S. Pat. No. 5,512,131, issued Apr. 30, 1996; U.S. patent application Ser. No. 09/004,583, filed Jan. 8, 1998, entitled “Method of Forming Articles including Waveguides via Capillary Micromolding and Microtransfer Molding,” by Kim, et al., now U.S. Pat. No. 6,355,198, issued Mar. 12, 2002; International Patent Application No. PCT/US96/03073, filed Mar. 1, 1996, entitled “Microcontact Printing on Surfaces and Derivative Articles,” by Whitesides, et al., published as WO 96/29629 on Jun. 26, 1996; International Patent Application No.: PCT/US01/16973, filed May 25, 2001, entitled “Microfluidic Systems including Three-Dimensionally Arrayed Channel Networks,” by Anderson, et al., published as WO 01/89787 on Nov. 29, 2001; U.S. patent application Ser. No. 11/246,911, filed Oct. 7, 2005, entitled “Formation and Control of Fluidic Species,” by Link, et al., published as U.S. Patent Application Publication No. 2006/0163385 on Jul. 27, 2006; U.S. patent application Ser. No. 11/024,228, filed Dec. 28, 2004, entitled “Method and Apparatus for Fluid Dispersion,” by Stone, et al., published as U.S. Patent Application Publication No. 2005/0172476 on Aug. 11, 2005; International Patent Application No. PCT/US2006/007772, filed Mar. 3, 2006, entitled “Method and Apparatus for Forming Multiple Emulsions,” by Weitz, et al., published as WO 2006/096571 on Sep. 14, 2006; U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007; and U.S. patent application Ser. No. 11/368,263, filed Mar. 3, 2006, entitled “Systems and Methods of Forming Particles,” by Garstecki, et al. Also incorporated herein by reference are U.S. Provisional Patent Application Ser. No. 60/920,574, filed Mar. 28, 2007, entitled “Multiple Emulsions and Techniques for Formation,” by Chu, et al.; U.S. Provisional Patent Application Ser. No. 61/255,239, filed Oct. 27, 2009, entitled “Droplet Creation Techniques,” by Weitz, et al.; U.S. Provisional Patent Application Ser. No. 61/239,402, filed Sep. 2, 2009, entitled “Multiple Emulsions Created Using Junctions,” by Weitz, et al.; and U.S. Provisional Patent Application Ser. No. 61/239,405, filed Sep. 9, 2009, entitled “Multiple Emulsions Created Using Jetting and Other Techniques,” by Weitz, et al. In addition, U.S. Provisional Patent Application Ser. No. 61/314,841, filed Mar. 17, 2010, entitled “Melt Emulsification,” by Shum, et al. is incorporated herein by reference.

The following examples are intended to illustrate certain embodiments of the present invention, but do not exemplify the full scope of the invention.

### Example 1

This example presents micro fluidic melt emulsification for encapsulation and release of certain species, in accordance with certain embodiments of the invention.

Double emulsions are structures comprising droplets of a first (inner) phase contained within larger droplets of a second (outer) phase, which is typically immiscible with the first phase, which in turn are contained within a continuous phase. Double emulsions are often used for encapsulation of species (or “active”) ranging from food additives such as nutrients and flavors, to components for personal care products, to drugs for therapeutic applications. Double emulsions may be thermodynamically unstable in some embodiments; for certain species to remain encapsulated within a double emulsion, surfactants are usually added to stabilize the double emulsion. With the addition of surfactants, the stability of double emulsions can be significantly enhanced; however, it can also become more difficult, in some cases, to destabilize double emulsions and release the species on demand for applications that require release of the species.

This example illustrates methods to selectively gel or harden the outer fluid of a double emulsion droplet to create a solid capsule, which may be used to surround or encapsulate an active contained in an inner fluid within the double emulsion droplet. In this example, a temperature-sensitive poly(N-isopropylacrylamide) (PNIPAM) gel is used for this purpose, although other materials may be used in other embodiments. Since PNIPAM switches between a swollen and a shrunken state at different temperatures, species encapsulated by PNIPAM can be released by changing the temperature. Another strategy is to solidify the outer phase of the double emulsion droplet by lowering the temperature so that the outer phase undergoes a liquid-to-solid transition, e.g., forming a solid “shell” encapsulating the active (which may be contained within a liquid inner phase within the solid shell of the capsule; release of the species can then be achieved by heating the outer phase to melt the outer phase, allowing the species to escape from the capsule, e.g., via liquid diffusion. The outer phase can also be formed from a mixture of materials having different properties, such as melting temperatures, may also be used in some cases to allow manipulation of the release profiles of the species to achieve controlled release.

In this example, a microfluidic approach is described for fabricating solid capsules for encapsulation and triggered release of a species from the capsules. Monodisperse double emulsion droplets having a molten or liquid outer phase were prepared in a capillary microfluidic device and formed into solid capsules by solidified the outer phase upon cooling of the droplets. These capsules demonstrated that encapsulation of various species with different sizes, charges, polarity, and/or surface-activity could be achieved; in addition, the species could be released from the capsules by heating the capsules above the melting temperature of the shell phase. To encapsulate multiple species, a microfluidic device for forming double emulsions with multiple inner droplets was used, which could produce multi-compartment solid capsules. These capsules could be used, for example, for encapsulating mutually incompatible species, reactants, etc. For example, the species may be ones that can react, and encapsulation of the species within different compartments may be used to prevent or control their reaction.

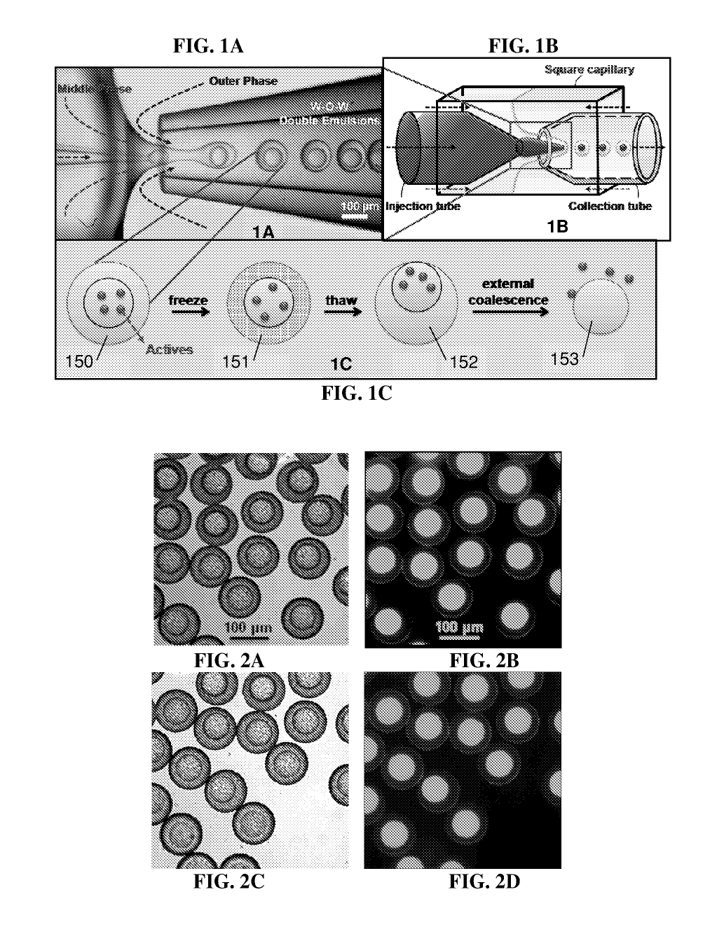

In a typical experiment, double emulsion droplets with a molten outer phases were prepared in a capillary microfluidic device as shown in FIG. 1A. The capillary microfluidic device was assembled by aligning two cylindrical capillaries coaxially inside a square capillary, as shown in FIG. 1B. The fluid for the inner phase was passed through a first cylindrical capillary or injection tube; and the outer phase was pumped through interstices between the outer square capillary and the injection tube in the same direction as inner phase fluid. The continuous phase fluid flowed into the square capillary from the opposite end of the inner and outer phases. For double emulsion droplets to form, the molten outer phase may be selected to be essentially immiscible with both the inner and continuous fluids, at least in this example. The continuous phase hydrodynamically flow focuses the inner and outer phases when they meet at the entrance of the second cylindrical capillary, or collection tube.

Double emulsion droplets were formed inside the collection tube, as shown in FIG. 1A. The continuous phase comprised water, glycerol, and PVA. The outer phase was a molten oil phase. The inner phase contained water with glycerol and various species. The double emulsion droplets were then cooled below the melting temperature of the encapsulating shell (outer) phase to form capsules. Agents encapsulated within the capsules, e.g., within the inner phase can be released on-demand by heating the capsules to a temperature that causes the outer phase to melt or liquefy; the concept of this approach is summarized in FIG. 1C.

In FIG. 1C, 150 shows a double emulsion droplet prepared in a microfluidic device. The outer phase undergoes a liquid-to-solid phase transition after cooling to form solid capsules shown in 151. By heating the capsules above the melting point of outer phase, the outer shell of the capsules could be thawed, thereby forming the double emulsion droplets shown in 152. As a result, the inner phase inside the melted shell can move freely or escape; since surfactants can be deliberately omitted from the outer phase, at least in some cases, species contained within the inner phase could be released due to coalescence of the inner phase with the continuous phase, as shown in 153.

The concept was demonstrated in this example using a water-in-oil-in-water (W-O-W) double emulsion to prepare shells of fatty acid glycerides for encapsulating a model encapsulant, FITC-dextran, which is fluorescently labeled with FITC (fluorescein isothiocyanate). A continuous phase of water with glycerol and poly(vinyl alcohol) (PVA), an outer phase of molten fatty glycerides (SUPPOCIRE AIM®, Gattefosse, melting point of 33° C. to 35° C.), and an inner phase of water-glycerol mixture with certain model species were used. Since the viscosity of the molten fatty acid glycerides is higher than that of pure water, limiting the range of flow rates at which double emulsion droplets could be prepared, glycerol was added to the inner and continuous phases to increase their respective viscosities. PVA was also added to the continuous phase to stabilize the double emulsion. The double emulsion droplets prepared in the microfluidic device were collected into a vial that was cooled in a bath of ice-water to speed the solidification of the outer phase to form solid shells.

FITC-dextran was encapsulated inside the capsules without leakage to the continuous phase, as shown in FIGS. 2A and 2B. FIG. 2A shows a bright field microscope image of double emulsions having solid shells of fatty acid glycerides. The continuous phase comprised water with 47.5 wt % glycerol and 5 wt % PVA. The outer phase of the droplets comprised molten fatty acid glycerides. The inner phase comprised water with 50 wt % glycerol and 0.2 wt % FITC-Dextran. FIG. 2B is a fluorescence microscope image of the same area as in FIG. 2A. These capsules remained stable at room temperature for at least six months and showed no observable leakage, as demonstrated by absence of fluorescence outside the capsules in FIGS. 2C and 2D after six months. In particular, FIG. 2C shows a bright-field microscope image of capsules having solid shells of fatty acid glycerides after the capsules were stored at room temperature for 6 months, and FIG. 2D is a fluorescence microscope image of the same area as in FIG. 2C.

Apart from achieving capsule stability, this technique also allows species to be released on demand by heating the capsules above the melting temperature of the outer phase. To facilitate the release of species, the surfactants can be deliberately omitted from the outer phase so that coalescence between the inner phase and the continuous phase occurs rapidly after melting of the outer shell.

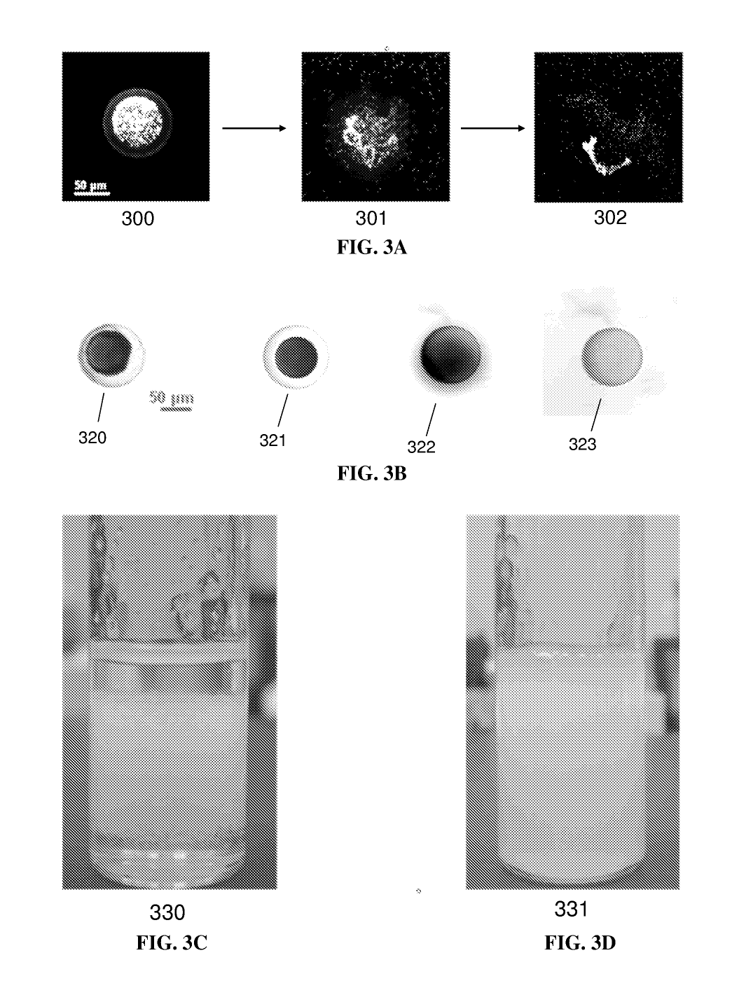

This simple release mechanism was demonstrated in various experiments by releasing 1-micrometer fluorescent latex beads encapsulated inside fatty acid glyceride capsules by heating the capsules to 37° C. The solid fatty acid glyceride shells gradually melted and, after heating for about 5 minutes, the outer shells underwent a solid-to-liquid transition. The double emulsion droplets burst, leading to the release of the latex beads from within the inner phase, as shown in FIG. 3A. This figure shows fluorescence microscope images showing the release of fluorescent beads from capsules of fatty acid glycerides. 300 shows solid capsules of fatty acid glycerides with fluorescence beads encapsulated within the capsules at room temperature; when the capsules were heated to 37° C., the fluorescence beads were released from the inner phase as shown in 301; after 5 minutes of heating, the fluorescence beads were almost completely released as shown in 302.

The same approach was also applied to solid capsules of paraffin oil (Wako, m.p. 42-44° C.), nonadecane (Sigma-Aldrich, m.p. 32° C.), and eicosane (Sigma-Aldrich, m.p. 37° C.). In all of these cases, the solid capsules demonstrated similar performance as the capsules of fatty acid glycerides. For example, FIG. 3B shows bright field images showing the release of toluidine blue from capsules of paraffin. 320 shows capsules of paraffin encapsulating toluidine blue at room temperature; when the capsules were heated to 45° C., the paraffin shell melted to form a liquid as shown in 321; toludine blue dye within the interiors of the capsules were released as shown in 322. The toluidine blue dyes were almost entirely released after 5 minutes of heating, as shown in 323.

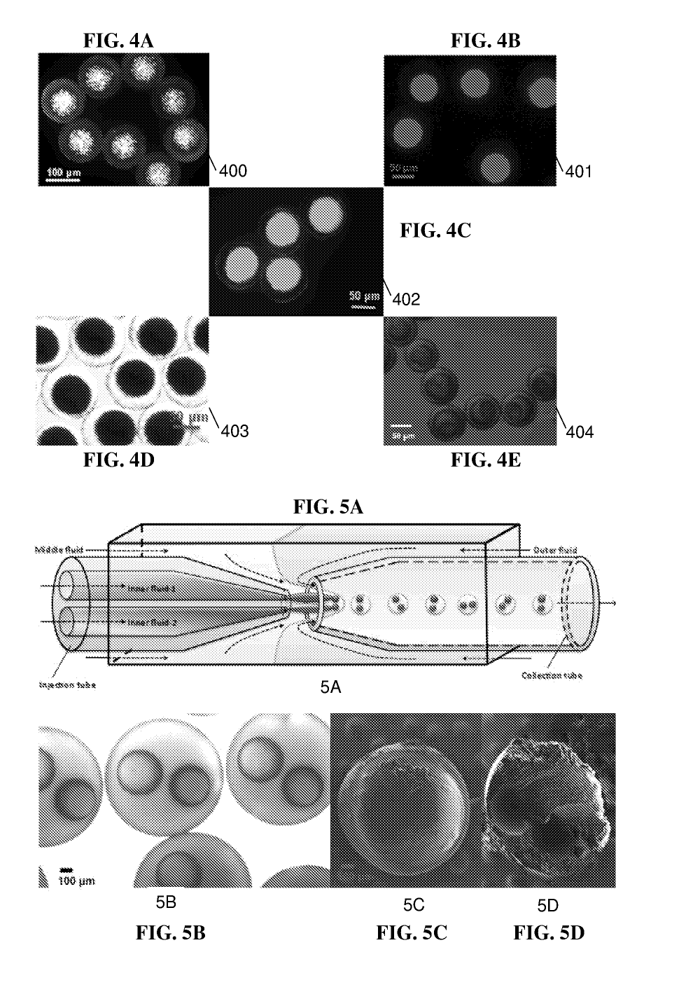

This method can also be applied to other species, e.g., having different sizes and/or charges. To demonstrate this, two positively charged dyes, rhodamine B and toluidine blue, and a negatively charged dye, fluorescein sodium salt, were used as model species in another set of experiments; these molecules are smaller than the FITC-dextran and fluorescent beads described above. In all of these cases, the dyes were essentially completely encapsulated in solid capsules of fatty acid glycerides, as shown in 401 (rhodamine B), 402 (fluorescein sodium salt), and 403 (toluidine blue) (FIGS. 4B-4D). For comparison, 400 (FIG. 4A) shows encapsulated fluorescent beads. The species were released by heating the capsules, as shown in FIG. 3B. The successful encapsulation and release of these smaller dyes highlights the low permeability of the solid capsules, and the effectiveness of the simple release mechanism.

These methods also appeared effective for the encapsulation of amphiphilic agents such as surfactants, which are typically very challenging to encapsulate using an emulsion approach. Without wishing to be bound by any theory, surfactants tend to adsorb at the interfaces of emulsions and cause the emulsions to destabilize. Using methods described herein, a concentrated laundry detergent (Unilever), which contained a mixture of bleach and different surfactants, was encapsulated as shown in 404 for laundry detergent (FIG. 4E). These methods differed from conventional techniques since the encapsulating outer phase solidified before the surfactants in the laundry detergent destabilized the emulsions, making it possible to encapsulate surface-active amphiphilic species. To test whether the laundry detergent remained encapsulated, the solid capsules were mixed with hexadecane. In the case of essentially perfect encapsulation, essentially no surfactant should be present outside the capsules in the continuous phase; thus, because essentially no surfactant was present, the hexadecane forms a layer floating on top of the capsules, as shown in 330 (FIG. 3C) since hexadecane is essentially immiscible in the continuous phase (water with glycerol and PVA). However, after heating the capsules at 37° C. for 5 minutes, the laundry detergent was released from the capsules and the surfactant within the laundry detergent was able to emulsify the hexadecane layer above the capsules, resulting in a cloudy mixture as shown in 331 (FIG. 3D), as the surfactant allowed an emulsion of the hexadecane in the continuous phase to form, resulting in the cloudy appearance. This result demonstrated the effectiveness of the methods for encapsulation and release of amphiphilic species.

This is illustrated in FIGS. 3C and 3D, showing bright field images showing the release of laundry detergent from capsules of fatty acid glycerides for emulsification of hexadecane. In 330, the top clear layer is hexadecane, and the cloudy layer below comprises capsules encapsulating laundry detergent. The continuous phase of the bottom layer is a solution of water containing glycerol and PVA. After heating at 37° C. for 5 minutes, the capsules melted, releasing the detergent to the continuous phase. The released detergent emulsified the hexadecane, resulting in a cloudy solution as shown in 331.

To quantify the stability during storage and the release efficiency of agents, an herbicide, dicamba (3,6-dichloro-2-methoxybenzoic acid) (BASF), was encapsulated in solid capsules and the release of dicamba into the surrounding continuous phase was monitored using a spectrophotometer. After about a month, only 5.73% of the dicamba was released from solid capsules of fatty acid glycerides, while 2.93% was released from solid capsules of paraffin. Additional experiments demonstrated that despite good encapsulation stability, the species could be released rapidly upon a suitable trigger. In particular, after heating at 37° C. for 5 minutes, 76.8% of dicamba was released from the capsules of fatty acid glycerides while 55.8% of dicamba was released from the paraffin shell after heating each of these at 45° C. for 5 minutes. These results showed that some of the methods disclosed herein combine the potential for extended shelf life with efficient on-demand release of agents.

One of the challenges for the encapsulation of certain species is to co-encapsulate multiple incompatible species in the same encapsulating structures, e.g., for specific synergistic effects, or for further chemical reactions upon release of the species from the capsule or particle. In this context, “incompatible” generally refers to species that may react when they are directly exposed to each other, in some cases spontaneously; in many cases, such reaction may be undesired before a certain point in time, e.g., prior to a triggering event. Generally, to prevent degradation or premature reaction, the species should not be pre-mixed before triggered release from the capsules or particles; thus, such species should be separated during the formation of the capsules. To accomplish this in this example, a glass capillary device, which has an injection tube with two separate internal channels, was designed to fabricate capsules having two internal compartments using a double emulsion approach, such that two incompatible species could be separately stored in each of the two internal compartments. Two streams of fluids with two different species could flow separately into the device through the two channels, as shown in FIG. 5A. The injection tube has two separate internal channels, which allow two different fluids to enter the devices separately. This method was demonstrated using two different dyes, Wright stain and rhodamine B, as model incompatible species for encapsulation in the solid capsules; the light (Wright) and dark dyes (rhodamine B) flowed in separate channels and formed two separate droplets without mixing, as shown in FIGS. 5B, 5C, and 5D. FIG. 5B shows a bright field microscope image of the solid capsules with two inner compartments containing aqueous solutions of Wright stain (light) and rhodamine B (dark); FIG. 5C shows a SEM image of a particle from FIG. 5B showing the surface of a dried capsule, while FIG. 5D shows a SEM image of a particle from FIG. 5B showing the cross-section of the capsule.

Capsules containing more than one species encapsulated therein may be promising, for example, as multi-functional capsules or as micro-reactors. By tuning the separation distance between the compartments containing the species, it is possible to manipulate the release profile of the species that are encapsulated. For example, if the two compartments are separated far enough apart from each other within the capsule, the two incompatible species can be released to the continuous phase separately, making these capsules useful for applications that require the simultaneous release of incompatible species. However, as another example, if the two compartments containing the species are positioned relatively close to each other within the capsule, the compartments can coalesce with each other and/or the species may become exposed to each other, before being released to the surroundings. These capsules can thus act, for example, as micro-reactors in which mixing of reactants is triggered by heating.

In summary, this example shows various techniques that use micro fluidic double emulsion droplets to fabricate capsules for the encapsulation and release of various species. By using a shell phase that undergoes a liquid-to-solid phase transition, certain double emulsion droplets can be converted to solid capsules with good encapsulation efficiency and/or stability. In addition, in some cases, species that are contained within the solid capsules can be released relatively rapidly, for example, when the capsules are heated above the melting point of the outer phase or shells of the capsules.

This example also shows encapsulation of amphiphilic species, which may, in some cases, otherwise destabilize emulsions and/or prevent encapsulation. In some experiments, capsules were fabricated having various compartments for encapsulating multiple species; such capsules may be useful in some embodiments for separately encapsulating incompatible or reactive species.

### Experimental

Materials: Materials used to prepare the continuous phase were water (18.2 MΩ·cm⁻¹ (megohm/cm), Millipore Milli-Q system), glycerol (EMD Chemicals Inc.), poly(vinyl alcohol) (PVA; M_{w}: 13,000-23,000 g·mol⁻¹, 87-89% hydrolyzed, Sigma-Aldrich Co.). Outer phase oils that were used included SUPPOCIRE AIM® oil (a mixture of glycerides of saturated fatty acids from C₈-C₁₈, m.p. 33-35° C., Gatefosse), Paraffin (CₙH₂ₙ₊₂, m.p. 42-44° C., Wako Pure Chemical Industries, Ltd.), nonadecane (Sigma-Aldrich Co.) and eicosane (Sigma-Aldrich Co.). Various model species for encapsulation used in these experiments included fluorescent beads (1 micrometer yellow-green fluorescent, fluorescent sulfate microspheres, Invitrogen, Inc.), fluorescein isothiocyanate-dextran (FITC-dextran, M_{w}: 10,000 g·mol⁻¹, Sigma-Aldrich Co.), fluorescein sodium salt (Sigma-Aldrich Co.), toluidine blue (Fluka), rhodamine B (Sigma-Aldrich Co.), dicamba (BASF), Wright stain (Sigma-Aldrich Co.), and commercially-available laundry detergent (Unilever).

Microfludics: Monodisperse w/o/w double emulsions were prepared using glass capillary-based microfluidic devices using known techniques (see, for example, International Patent Application Serial No. PCT/US2008/004097, filed Mar. 28, 2008, entitled “Emulsions and Techniques for Formation,” by Chu, et al., published as WO 2008/121342 on Oct. 9, 2008; International Patent Application No. PCT/US2006/007772, filed Mar. 3, 2006, entitled “Method and Apparatus for Forming Multiple Emulsions,” by Weitz, et al., published as WO 2006/096571 on Sep. 14, 2006; U.S. Provisional Patent Application Ser. No. 61/160,020, filed Mar. 13, 2009, entitled “Controlled Creation of Emulsions, Including Multiple Emulsions,” by Weitz, et al.; U.S. Provisional Patent Application Ser. No. 61/255,239, filed Oct. 27, 2009, entitled “Droplet Creation Techniques,” by Weitz, et al.; U.S. Provisional Patent Application Ser. No. 61/239,402, filed Sep. 2, 2009, entitled “Multiple Emulsions Created Using Junctions,” by Weitz, et al.; and U.S. Provisional Patent Application Ser. No. 61/239,405, filed Sep. 9, 2009, entitled “Multiple Emulsions Created Using Jetting and Other Techniques,” by Weitz, et al., each of which is incorporated herein by reference).

The continuous phase of each encapsulation process was a mixture of water and glycerol mixed in a one-to-one weight ratio with 5 wt % PVA. The inner phases in various experiments included (1) water, glycerol, and FITC-Dextran (49.9, 49.9, and 0.2 wt %); or (2) water, glycerol, and fluorescence beads (47.5 wt %, 47.5 wt %, and 5% vol); or (3) water, glycerol, and rhodamine B (49.97, 49.97, and 0.06 wt %); or (4) water, glycerol, and fluorescein sodium salt (49.995, 49.995, and 0.01 wt %); (5) water, glycerol, and toluidine blue (49.75, 49.75, and 0.5 wt %); (6) water and Wright stain (99 and 1 wt %); and (7) water and rhodamine B (99.5 and 0.5 wt %). During fabrication of the double emulsion with the fatty acid glycerides, a typical set of flow rates for the continuous, outer, and inner phases was 12,000, 1,500, and 200 microliters/hr, respectively; with paraffin oil, the flow rates of the continuous, outer, and inner phases were 10,000, 1,200 and 700 microliters/hr respectively. In the preparation of double emulsion with two inner droplets, a typical set of flow rates for the continuous, outer, and the two inner phases was 30,000, 7,000, and 700 (rhodamine B)-800 (Wright stain) microliters/hr, respectively. All fluids were pumped into the capillary microfluidic device using syringe pumps (Harvard PHD 2000 series).

Sample characterization: The microfluidic process was monitored using an inverted optical microscope (DM-IRB, Leica) fitted with a fast camera (Phantom V9, Vision Research). Bright-field and fluorescence images were obtained with 10× objectives at room temperature using an automated inverted microscope with fluorescence (Leica, DMIRBE) equipped with a digital camera (QImaging, QICAM 12-bit). The release profile of Dicamba was monitored using a UV-vis spectrophotometer (Nanodrop, ND 1000). Scanning electron microscopic (SEM) images of dried capsules coated with a thin layer of platinum and palladium were taken using a Zeiss Supra 55VP field emission scanning electron microscope (FESEM, Carl Zeiss, Germany) at an acceleration voltage of 20 kV.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a particle having an average diameter of less than about 1 mm, the particle comprising an at least partially solid outer phase and an inner phase, the at least partially solid outer phase partially or completely encapsulating the inner phase, wherein the at least partially solid outer phase has a melting temperature greater than about 0° C.

2. The article of claim 1, wherein the inner phase is essentially free of auxiliary stabilizing agents.

3. The article of claim 1, wherein the at least partially solid outer phase is a semisolid.

4. The article of claim 1, wherein the at least partially solid outer phase comprises a solid and a liquid.

5. The article of claim 1, wherein the at least partially solid outer phase is completely solid.

6. The article of claim 1, wherein the inner phase comprises a liquid.

7. The article of claim 1, wherein the inner phase comprises a solid.

8. The article of claim 1, wherein the inner phase comprises a semisolid.

9. The article of claim 1, wherein the particle further comprises a species.

10. The article of claim 9, wherein the species comprises a nanoparticle.

11-13. (canceled)

14. The article of claim 9, wherein the species is amphiphilic.

15. (canceled)

16. The article of claim 1, wherein the at least partially solid outer phase is immiscible in water.

17. The article of claim 1, wherein the at least partially solid outer phase is immiscible with water.

18. The article of claim 1, wherein the at least partially solid outer phase has a melting temperature greater than 10° C.

19-20. (canceled)

21. The article of claim 1, wherein the at least partially solid outer phase comprises paraffin oil.

22. The article of claim 1, wherein the inner phase is a first inner phase, the particle further comprising a second inner phase distinguishable from the first inner phase.

23. A method, comprising:
providing a first fluid and a second fluid, the second fluid comprising a species, wherein the first fluid and the second fluid are at least partially immiscible;
surrounding at least portion of the second fluid with the first fluid to form a multiple emulsion; and
solidifying at least a portion of the first fluid to form a capsule, wherein at least 90% of the species is partially or completely encapsulated within the capsule.

24. The method of claim 23, wherein the first fluid has a melting temperature greater than 0° C.

25. The method of claim 23, wherein the first fluid has a melting temperature greater than 10° C.

26-27. (canceled)

28. The method of claim 23, wherein the species is amphiphilic.

29. The method of claim 23, wherein the species comprises a nanoparticle.

30. The method of claim 23, wherein solidifying at least a portion of the first fluid comprising cooling the multiple emulsion to at least a temperature sufficient to initiate a phase change in at least a portion of the first fluid.

31. The method of claim 23, wherein the capsule is suspended within a continuous fluid.

32-34. (canceled)

35. A method, comprising:
providing a droplet having an average diameter of less than about 1 mm, the droplet comprising an outer phase and an inner phase, the outer phase partially or completely encapsulating the inner phase, wherein the outer phase has a melting temperature greater than 0° C.; and
solidifying at least a portion of the outer phase by altering the temperature of the droplet to produce a capsule.

36. The method of claim 35, wherein the droplet further comprises a species.

37. The method of claim 36, wherein the species comprises nanoparticles.

38-39. (canceled)

40. The method of claim 35, wherein the at least a portion of the outer phase that is solidified by altering temperature is liquefiable by altering its temperature.

41. The method of claim 35, wherein the outer phase has a melting temperature greater than 20° C.

42. (canceled)

43. The method of claim 35, wherein the outer phase is immiscible in water.

44. The method of claim 35, wherein the inner phase is an aqueous solution.

45. A method, comprising:
providing a particle having an average diameter of less than about 1 mm, the particle comprising an at least partially solid outer phase and an inner phase, the at least partially solid outer phase partially or completely encapsulating the inner phase, wherein the at least partially solid outer phase has a melting temperature greater than 0° C.; and
releasing a species from the particle by melting the at least partially solid outer phase.

46. (canceled)

47. The method of claim 45, wherein the species comprises a nanoparticle.

48. The method of claim 45, wherein the threshold temperature is at least 0° C.

49-53. (canceled)

54. An article, comprising:
a particle having a shell surrounding at least one liquid core, the particle having an average diameter of less than about 1 mm, the shell having a melting temperature greater than about 0° C.

55. The article of claim 54, wherein the particle comprises at least a first liquid core and a second liquid core distinguishable from the first liquid core.

56-58. (canceled)

59. The article of claim 9, wherein the species comprises a pesticide.
